(19) United States
(12) Patent Application Publication
(10) Pub. No.: US 2026/0228886 A1
(43) Pub. Date: Aug. 6, 2026

(54) SYSTEM AND METHOD FOR ANALYSIS OF MEDICAL IMAGES

(71) Applicant: NAITIVE TECHNOLOGIES LIMITED, Cambridge (GB)

(72) Inventor: Christopher TROMANS, Cambridge (GB)

(73) Assignee: NAITIVE TECHNOLOGIES LIMITED, Cambridge, CA (US)

(21) Appl. No.: 19/151,051

(22) PCT Filed: Jan. 25, 2024
(86) PCT No.: PCT/IB2024/050708

§ 371 Date: Jul. 25, 2025

(30) Foreign Application Priority Data
Jan. 25, 2023 (GB) 2301075.4

(51) Int. Cl. G06T 7/00

(52) U.S. Cl. CPC G06T 7/0012; G06T 2207/10081; G06T 2207/10088; G06T 2207/10116; G06T 2207/20081; G06T 2207/20084; G06T 2207/20124; G06T 2207/30008

(57) ABSTRACT

A method and system for analysis of medical images is presented in the field of analysis of medical images. The method and systems is provided to determine one or more parameters of bones in medical images. The method and system may be characterized as an advancement in analysis of medical images to ascertain bone health and discern pathologies in bone. Specifically, a system and method is disclosed for analysing medical images showing at least a portion of a bone to determine one or more bone parameters for the at least one portion of a bone. Steps taken by the system and method include: determining a region of interest of the medical image; identifying a boundary of at least one of an inner edge and an outer edge of cortical bone of the at least one portion of the bone within the region of interest; identifying at least one boundary of trabecular bone of the at least one portion of the bone within the region of interest; utilising the identified boundaries to determine at least one of the following bone parameters: orientation, bone mineral density, length, width of trabecular structure, for the at least one portion of bone within the region of interest.

### FIELD OF THE INVENTION

This invention is in a general field of analysis of medical images. In particular the invention relates to analysis to determine one or more parameters of bones in medical images. The invention may be characterized as an advancement in analysis of medical images to ascertain bone health and discern pathologies in bone.

### BACKGROUND

Strong, healthy bones are a fundamental component of overall bodily health. Weakened bones increase the risk of serious fracture, particularly affecting the increasingly ageing population. Reduced bone strength results primarily from a number of inter-related metabolic bone pathologies, including (but not limited to): osteoporosis, osteopenia, osteomalacia, Paget's disease of the bone, which may arise for a number of reasons. The earlier such pathologies are detected, the better the prognosis for the patient., For example, osteoporosis can be reversed with widely-available and cheap medication.

Bone is a complex living structure, primarily comprised of two types of tissue: cortical bone and trabecular bone. To measure or detect a particular structure or tissue in images of complex living structures an Active Shape Model (ASM) may be used as disclosed by T. Cootes and R. Baldock, “Model-based methods in analysis of Biomedical images,” in Image Processing and Analysis: A Practical approach, United Kingdom: Oxford University Press, 1999.

The composition and construction of cortical and trabecular bone, and the interfaces between them, enables the skeleton to perform its essential mechanical functions with reduced risk of fracture, pain, and disease. Cortical bone makes up about 80% of bone mass, and forms a hard outer layer that is dense, strong, and durable. Trabecular bone makes up about 20% of bone mass and has a honeycomb-like structure comprised of individual trabeculae. It transfers mechanical loads from articular surfaces to the cortical bone and essentially acts as a biological shock absorber.

As one example: it is estimated that over 200 million people worldwide suffer from osteoporosis. Osteoporosis adversely affects bone quantity, bone quality and bone morphometry. It is a progressive, systemic skeletal disorder, characterised by a loss of bone mass, deterioration of bone microarchitecture and increased bone fragility. Such changes vary from person to person, with individuals exhibiting different patterns of bone loss as disclosed by P. Choksi, K. J. Jepsen, and G. A. Clines, “The challenges of diagnosing osteoporosis and the limitations of currently available tools,” Clinical Diabetes and Endocrinology, vol. 4, no. 1, p. 12, December 2018, doi: 10.1186/s40842-018-0062-7.

As a second example, a staggering 1 in 3 women and 1 in 5 men over the age of 50 can expect to experience a fragility fracture in their lifetime. Worse, after a first fracture, patients are at a nearly two-fold increased risk of a subsequent fracture, yet 80% do not currently receive diagnostic testing.

Diagnostically, such pathologies have insidious onset, are progressive and “silent” until they are quite advanced. Initially, they present with few signs or symptoms to prompt care-seeking by patients and investigation by physicians. So, accepting that “The earlier such pathologies are detected, the better the prognosis for the patient”, the fundamental need is to detect evidence for likely pathology as early in its cycle of development as possible. That is what is provided in the current invention, based as it is on incidental findings: that is information extracted that is not directly related to the principal reason for doing the scan.

### Current Methods to Assess Bone Health

Bone health is assessed primarily using images, typically images based on x-rays. This is because, unlike MRI and ultrasound, x-rays can penetrate dense bone tissue, revealing its inner structure. A conventional plain-film x-ray image records the attenuation of the x-ray beam as it passes through tissue. There is more attenuation by trabecular bone than soft tissue; but less than through cortical bone. Plain-film x-ray images also show the scatter of x-rays as they pass through the object of interest.

Computed Tomography (CT) records low dose x-ray scans from many directions surrounding the object of interest (typically 720 0.5 degree scans) and enables construction of a 3D volume in which cortical bone, trabecular bone, and soft tissue are clearly visible. Such a 3D image is quantitative: each voxel records the x-ray density measured in Hounsfield units.

A number of technologies have also been developed that deploy multiple energies (e.g. Dual Energy X-Ray Absorptiometry (DEXA)) or phases, though the latter is currently mostly only available in research establishments. Dual Energy X-Ray Absorptiometry (DEXA) is the current gold-standard for measuring bone mineral density and diagnosing osteoporosis and the related condition osteopenia. It is a measure of bone quantity, and DEXA results are combined with other patient factors as part of fracture risk assessment.

During a Dual Energy X-Ray Absorptiometry (DEXA) scan, two X-Ray beams of different energy levels are aimed at a patient's bones. The difference in total absorption of the two beams is used to subtract out the absorption by soft tissue, leaving the absorption by bone, from which estimations of Bone Mineral Content (BMC) and Bone Mineral Density (BMD) are computed. BMD results are usually reported in terms of standard deviations from the mean of a young healthy population (T-score), whereas the associated Z-score corrects for age and sex. A T-score greater than −1 is considered normal, whilst a T-score between −1 and −2.5 is indicative of osteopenia, and a T-score less than −2.5 is indicative of osteoporosis. Z-score corrects for age and sex.

Though Dual Energy X-Ray Absorptiometry (DEXA) is often regarded as the “gold standard” for diagnosing bone pathologies, particularly osteoporosis, it has many limitations. A fundamental limitation is that DEXA is almost never applied until a bone pathology is suspected and confirmation is sought. This is, as noted above, most often late in the development of pathology.

Trabecular Bone Score (TBS) has been developed to provide some information about bone quality and susceptibility to fracture. TBS builds upon DEXA by providing information about bone microarchitecture as disclosed by D. Hans, E. Šteňová, and O. Lamy, “The Trabecular Bone Score (TBS) Complements DXA and the FRAX as a Fracture Risk Assessment Tool in Routine Clinical Practice. ,” Curr Osteoporos Rep, vol. 15, no. 6, pp. 521-531, 2017, doi: 10.1007/s11914-017-0410-z.

Trabecular Bone Score (TBS) quantifies local variations in grey-level of the greyscale spine DEXA image, as a surrogate for bone trabecular microarchitecture. Healthier bone has a well-structured trabecular bone and a higher TBS compared to an osteoporotic bone which has altered trabecular bone and a relative lower TBS. It is independently associated with fracture risk. However, as a measure of bone quality, TBS is not without its limitations. Owing to the poor resolution of DEXA images, TBS provides an impression, rather than direct visualisation, of trabecular microarchitecture. Despite the widespread use of Dual Energy X-Ray Absorptiometry (DEXA) and Trabecular Bone Score (TBS), a robust test that provides a more comprehensive description of bone strength is required. Due to expense and limited availability, DEXA is almost never applied until a bone pathology is suspected, for example because a patient has already suffered from injury of a bone. TBS does not take account of other determinants of bone quality, such as cortical microarchitecture, and other determinants of bone strength, such as morphometry.

Moreover, osteoporosis is a major risk factor for adverse outcomes, such as periprosthetic fracture and aseptic loosening. A robust test that integrates seamlessly into the preoperative assessment is required, to inform surgical decision making (including decision to operate, use of cemented vs uncemented prostheses, and prosthesis selection). Plain-film and digitally recorded X-Rays are a routine part of surgical planning and therefore such a test could fit seamlessly into existing clinical workflows without a requirement for additional imaging.

Though plain-film and digitally recorded x-ray images have a number of limitations, they enjoy a number of huge advantages. Among these are:
1. X-rays are the most commonly requested medical imaging examination;
2. X-ray machines are ubiquitous across primary and secondary care;
3. the average radiation dose from X-Ray is an order of magnitude less than the equivalent CT exam; and
4. X-Ray is more than an order of magnitude higher resolution than Dual Energy X-Ray Absorptiometry (DEXA) (e.g. a hip X-Ray is comprised of 1100 by 1100 pixels, whilst a hip DEXA is comprised of 250 by 300 pixels).

Assessing bone quality from images is not a new concept. In the 1970s Singh et al. developed an X-Ray based index for classifying trabecular bone loss at the proximal femur as disclosed by M. Singh, A. R. Nagrath, and P. S. Maini, “Changes in trabecular pattern of the upper end of the femur as an index of osteoporosis.,” J Bone Joint Surg Am, vol. 52, no. 3, pp. 457-67, April 1970. More recent developments artificial intelligence (AI) such as disclosed by R. Jang, J. H. Choi, N. Kim, J. S. Chang, P. W. Yoon, and C. H. Kim, “Prediction of osteoporosis from simple hip radiography using deep learning algorithm,” Scientific Reports, vol. 11, no. 1, December 2021, doi: 10.1038/s41598-021-99549-6.

The Singh Index is based on a simplified representation (and disappearance) of specific trabecular groups. However, there have been cases of poor intra-and inter-observer agreement and a lack of correlation with Bone Mineral Density (BMD) with the Singh Index as disclosed by F. Mir, I. Nazir, and M. Naseed, “Comparison of Radiographic Singh Index with Dual-Energy X-Ray Absorptiometry Scan in Diagnosing Osteoporosis,” Matrix Science Medica, vol. 5, no. 1, p. 17, 2021, doi: 10.4103/MTSM.MTSM_41_20. Therefore the Singh Index is not used much in clinical practice.

### Unmet Needs

Current pathways for identifying and characterising osteoporosis rely on physicians identifying at-risk patients (primary prevention), or fracture liaison services identifying patients who have already had a fragility fracture (secondary prevention) and referring them for a Dual Energy X-Ray Absorptiometry (DEXA) scan. With three-quarters of cases of osteoporosis undiagnosed, and the growing burden of fragility fractures and periprosthetic fractures, diagnostic pathways for osteoporosis are not fit for purpose.
Unmet need 1: The current pathways fail to identify the majority of cases of osteoporosis, and traditional opportunities to identify cases (e.g. physician's office visits) are diminishing. A new approach to osteoporosis case-finding is required.
Unmet need 2: Dual Energy X-Ray Absorptiometry (DEXA) measures Bone Mineral Density (BMD), which explains less than 50% of bone strength. Most patients who experience a fragility fracture have a BMD value in the osteopenic range and are not osteoporotic as is discussed by S. Williams, L. Khan, and A. A. Licata, “DXA and clinical challenges of fracture risk assessment in primary care,” Cleveland Clinic Journal of Medicine, vol. 88, no. 11, pp. 615-622, November 2021, doi: 10.3949/ccjm.88a.20199. Although Trabecular Bone Score (TBS) provides some information about bone quality, a robust test that provides a more comprehensive description of bone strength and fracture risk is required.
Unmet need 3: Dual Energy X-Ray Absorptiometry (DEXA) and Trabecular Bone Score (TBS) provide limited information about a patient's unique bone structure or pattern of bone loss, whilst osteoporosis drugs exhibit distinct patterns of protection. Effects on Bone Mineral Density (BMD) explain only 48-63% of fracture risk reduction conveyed by these drugs. A robust test that facilitates a treat-to-target (precision medicine) approach to osteoporosis prescribing is required.
Unmet need 4: Osteoporosis screening is not part of the current preoperative assessment for joint arthroplasty or other surgical procedures, for example an implant that may require the use of surgical nails. Yet osteoporosis is a major risk factor for adverse outcomes, such as periprosthetic fracture and aseptic loosening. A robust test that integrates seamlessly into the preoperative assessment is required, to inform surgical decision making (including decision to operate, use of cemented vs uncemented prostheses, and prosthesis selection).

### SUMMARY OF THE INVENTION

According to the invention there is provided a system and method of analysing medical images showing at least a portion of a bone to determine one or more bone parameters for the at least one portion of a bone comprising the steps of: determining a region of interest of the medical image; identifying a boundary of at least one of an inner edge and an outer edge of cortical bone of the at least one portion of the bone within the region of interest; identifying at least one boundary of trabecular bone of the at least one portion of the bone within the region of interest; utilising the identified boundaries to determine at least one of the following bone parameters: orientation, bone mineral density, length, width of trabecular structure, for the at least one portion of bone within the region of interest.

The bone parameters may be used to inform predictions of bone strength or risk of bone fracture. The bone parameters may be used to inform characterization of presence of osteoporosis or osteopenia development in the bone or of risk of development of osteoporosis or osteopenia in the bone in the future.

Preferably, the medical image is one of: an X-ray image, a CT scan, or an MR image.

Preferably, after the outer boundary of the cortical bone has been identified, performing a further segmentation step on the outer boundary.

Preferably, the method further comprises the step of segmenting the region of cortical bone contained within the identified boundaries, within the region of interest.

According to the system and method of analysing medical images, the segmentation of the region of cortical bone may analyze the medical image at different spatial resolutions to detect low frequency changes in the medical image. Outputs of the segmentation step may be checked for shape acceptance against a known shape metric.

Preferably, the known shape metric is transformed to align with the segmented outer boundary.

Transformation of the known shape metric may comprise at least one of: rotation, translation, scaling, of the parameters of the known shape metric.

Preferably, the known shape metric comprises at least one of a shape loss metric, which relates a distance in the shape to a nearest edge in the segmentation; and an area loss metric, which is the proportion of zero values in the segmentation inside the fitted active shape.

The area loss metric may be computed as the ratio of the symmetric difference of the set of pixels in the transformed parameterised shape and the segmented area of the body part, to the total number of pixels in the segmented area.

Preferably, the shape loss metric is calculated as the total perpendicular distance between each of the control points of the transformed parameterised shape and the segmented area, normalised by the number of control points and image pixel spacing.

A threshold may be applied to the known shape metric to identify images for which the bone segmentation is not within an allowed tolerance.

The system and method of analysing medical images may further comprise the step of determining the area of the at least a portion of bone in the region of interest that is occupied by cortical bone.

Preferably, the method further comprises the step of determining the ration of cortical to trabecular bone in the at least one portion of bone in the region of interest. The at least one portion of bone may comprise at least one of: femur, vertebrae, ankle, wrist, clavicle, or mandible.

Further preferably, the ratio of cortical to trabecular bone is used to determine one or more of the at least one bone parameters.

The system and method of analysing medical images may further comprise determining the relative intensity of the cortical bone.

Preferably, the outer boundary of the cortical bone is identified using the contrast between the edge of the at least one portion of bone and the surrounding area of the image.

A machine learning model may be used to identify the boundary of the cortical bone and/or the trabecular bone.

Preferably, one or more boundaries of the region of interest are determined using a machine learning model. The machine learning model may use at least one of a convolutional neural network or a multilayer perceptron.

Further preferably, the region of interest is a rectangular region.

The method may further comprise the step of determining a verification metric to assess the plausibility of the segmented shape in the region of interest compared to a database of predefined segmented shapes.

Preferably, the method further comprises a step of identifying imaging artefacts in the region of interest of the medical image. The artefacts may comprise one or more of: a surgical implant, a surgical nail, or other foreign body. The artefacts may arise from soft tissue, skin or body fat.

Preferably, a convolutional neural network is used to identify the one or more artefacts, and the medical image is further processed to remove the one or more artefacts from the region of interest of the medical image.

Preferably, the one or more bone parameters are used to determine one or more actionable metrics. The actionable metrics may comprise at least one of:
an indication of the presence of osteoporosis/osteopenia;
a classification of osteoporosis, osteopenia or healthy bone structure in line with that expected in the general population is a prediction of the severity of osteoporosis;
a risk of fracture in a given period;
a prediction of intra-operative fracture;
a proposal of a suitable bone implant type including a prediction of surgical outcomes, such as peri-prosthetic fracture and aseptic loosening following insertion of a bone implant.

In the system and method of analysing medical images, the actionable metric may be determined to take account of one or more of: age, sex, ethnicity, height, weight, BMI, smoking status, alcohol use, glucocorticoid use, prior fracture, age at menarche, age at menopause and family history. Further preferably, the actionable metric further comprises a set of individual bone health metrics from imaging examinations acquired at different points in time.

The foregoing summary is considered as illustrative of the principles of the invention. The invention will now be described, by way of examples, with reference to the accompanying figures in which:

### BRIEF DESCRIPTION OF THE FIGURES

FIG. 1 shows a direct visualisation, of trabecular microarchitecture and corresponding different Trabecular Bones Scores (TBS) for a healthy patient and an osteoporotic patient;

FIG. 2 shows an illustration of the Singh Index which shows the visual appearance of different Trabecular groups;

FIG. 3 shows an image processing pipeline for medical scan images:

FIG. 4 shows an example proximal femur region of interest (bounded by square of dashed line) in a pelvic X-Ray;

FIG. 5 shows example bone images from U-Net output on training data;

FIG. 6 shows segmentation of an image of a proximal femur;

FIG. 7 shows Active shapes fitted around a range of inner and outer cortical segmentations;

FIG. 8 shows an unsuccessful segmentation from a subject with skin folds.

### DETAILED DESCRIPTION OF THE INVENTION

Examples of the system and method of analysing medical images are presented that use explainable artificial intelligence (AI) powered technologies. The technologies are applied to medical scan images to identify different bone parameters. These parameters inform predictions of fracture risk and risk of osteoporosis. The medical scan image is one of: an X-ray image, a CT scan, or an MR image recorded digitally or on plain film, or another medical imaging technology.

To technically de-risk this approach, a suite of artificial intelligence (AI) or machine learning (ML) algorithms are used. The algorithms automate quantification of features of cortical and trabecular bone and to predict parameters of the bone from the medical images.

Suitable medical images of bones include but are not limited to images of bone including spine, vertebrae, hip, pelvis, leg, knee, ankle, foot, arm, wrist, clavicle or mandible. The parameter may be used to ascertain the health of the bones and discern pathologies in the bones which may be developing or prone to develop osteoporosis or osteopenia.

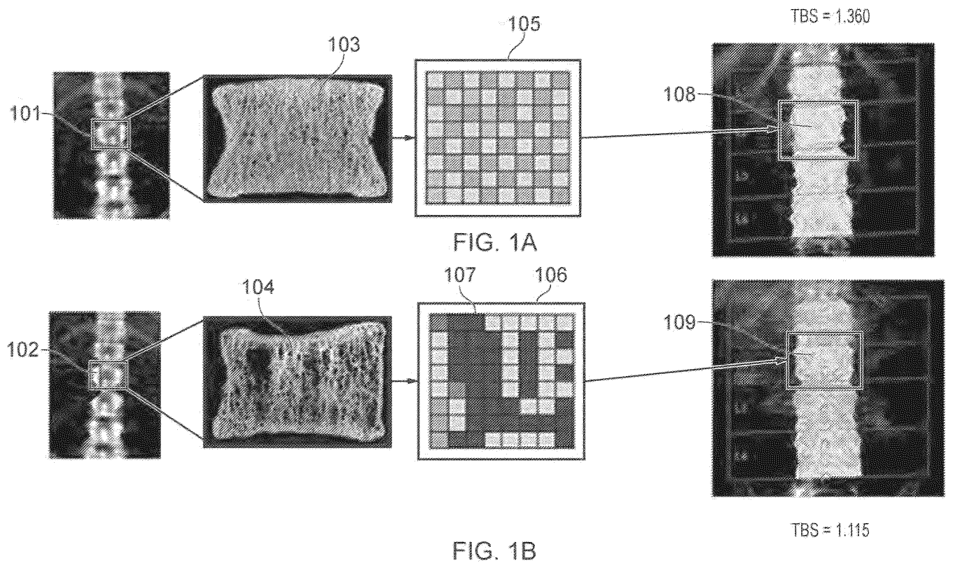

Trabecular Bone Score (TBS) provides an impression, rather than direct visualisation, of trabecular microarchitecture. FIG. 1 comprises FIG. 1A which shows well-structured trabecular bone in a healthy vertebra. It also comprises FIG. 1B which altered trabecular bone in an osteoporotic vertebra.

Left most in FIGS. 1A and 1B is a spinal vertebra 101, 102 from an X-ray image of a spine. FIG. 1A shows the spine of a healthy patient with the healthy vertebrae 101, and FIG. 1B shows the spine of an osteoporotic patient with the osteoporotic vertebrae 102.

Second from the left in FIGS. 1A and 1B are cross section views. A view second from the left in FIG. 1A shows a cross section 103 of the healthy vertebrae 101. A map 105 of the Bone Mineral Density (BMD) shows the BMD evenly distributed across the cross section 103 of the healthy vertebrae 101.

A view second from the left in FIG. 1B shows a cross section 104 of the osteoporotic vertebrae 102. A map 106 of the BMD shows a region 107 of loss of BMD from the cross section 104 of the osteoporotic vertebrae 102.

Right most in FIGS. 1A and 1B are Dual Energy X-Ray Absorptiometry (DEXA) images. Right most in FIG. 1A is a DEXA image 108 of the healthy vertebrae 101, and right most in FIG. 1B is a DEXA image 109 of the osteoporotic vertebrae 102. A Trabecular Bone Score (TBS) of 1.360 is shown for the healthy vertebrae 101, and a TBS of 1.115 is shown for the osteoporotic vertebrae 102.

Trabecular Bone Score (TBS) quantifies local variations in grey-level of the greyscale spine DEXA image, as a surrogate for bone trabecular microarchitecture. The TBS may be independently associated with fracture risk.

Although a variety of imaging modalities may be used, X-Ray images recorded digitally or on plain film is the preferred imaging modality as:
i) X-Ray is the most commonly requested medical imaging exam,
ii) X-Ray machines are ubiquitous across primary and secondary care,
iii) the average radiation dose from X-Ray is an order of magnitude less than the equivalent CT exam, and
iv) X-Ray is more than an order of magnitude higher resolution than Dual Energy X-Ray Absorptiometry (DEXA) (e.g. a hip X-Ray is comprised of 1100 by 1100 pixels, whilst a hip DEXA is comprised of 250 by 300 pixels).

The methods as described use the significantly higher resolution of X-Ray compared to DEXA, to extract and quantify one or more features of bone that are independently associated with bone strength, to identify osteoporosis and predict fracture risk. Our aim is to provide the most explainable and comprehensive description of bone strength and fracture risk.

An image processing pipeline for the analysis of medical scan images has been developed. This is described below. The images show the pelvic and hip region of a patient. Alternatively, the methods disclosed here apply equally to other relevant skeletal structures such as the spine, vertebrae, hip, pelvis, leg, knee, ankle, foot, arm, wrist, clavicle, or mandible or other bone or group of bones.

The system and method of analysing medical images is configured to use clinical images from the Picture Archiving and Communication System (PACS) medical imaging archives. However, other archiving systems or sources of clinical images may also be used.

In all cases, the methods have been developed and demonstrated on a large database of carefully-curated cases. Specifically, a proprietary patient discovery platform identified 509 patients for which matched datasets of pelvic x-ray images and Dual Energy X-Ray Absorptiometry (DEXA) scans were available and which were acquired within 6 months of each other. In total, a set of 1,469 matched pairs of images, from 509 patients, was identified, which were retrieved from the clinical PACS and pseudonymised for image and statistical analysis. For 109 patients, this dataset contained more than one hip or pelvic X-Ray taken at different times. Within this matched set, for the case of pelvic X-Rays, if both a right and left hip DEXA scan was available, the X-Ray was paired against each (since a pelvic X-Ray includes both hips). Any hip X-Rays with laterality that did not match the DEXA scan laterality, as well as DEXA scans with missing T-scores and any duplicate DEXA scans were removed. A further 463 X-Rays for which the radiographic view was not anterior-posterior (front-to-back) were also removed. This left a total of 931 matched pairs to be analysed to determine one or more bone parameters, including orientation, bone mineral density, length, width of trabecular structure, for the at least one portion of bone within the region of interest, and to determine bone strength.

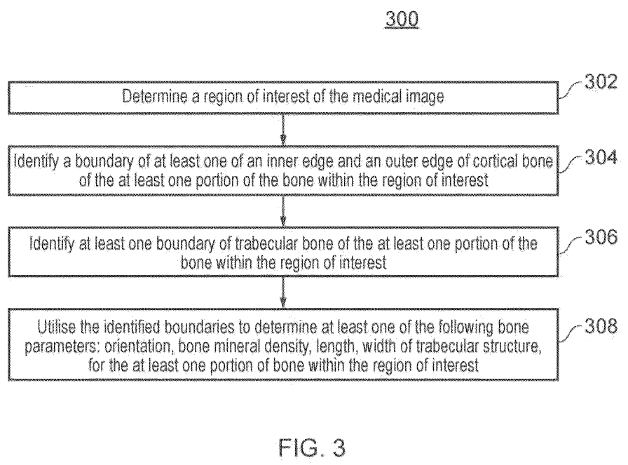

FIG. 3 shows a flowchart of an example method 300 of this system and method of analysing medical images. In some implementations, one or more process blocks of FIG. 3 may be performed by a device. As shown in FIG. 3, process 300 includes determining a region of interest of the medical image (block 302).

As also shown in FIG. 3, process 300 may include identifying a boundary of at least one of an inner edge and an outer edge of cortical bone of the at least one portion of the bone within the region of interest (block 304). After the outer boundary of the cortical bone has been identified, a further segmentation step on the outer boundary is performed. Preferably, the method also includes the step of segmenting the region of cortical bone contained within the identified boundaries, within the region of interest. Optionally the relative intensity of the cortical bone is also determined.

As further shown in FIG. 3, process 300 may include identifying at least one boundary of trabecular bone of the at least one portion of the bone within the region of interest (block 306).

As also shown in FIG. 3, process 300 may include utilising the identified boundaries to determine at least one of the following bone parameters: orientation, bone mineral density, length, width of trabecular structure, for the at least one portion of bone within the region of interest (block 308).

Although FIG. 3 shows example blocks of process 300, in some implementations, process 300 may include additional blocks, fewer blocks, different blocks, or differently arranged blocks than those depicted in FIG. 3. Additionally, or alternatively, two or more of the blocks of process 300 may be performed in parallel. Each of these stages of the method will now be described in more detail as follows.

Prior to the image analysis as described above, a medical scan image is acquired for analysis. The system and method of analysing medical images is configured to receive an image provided from an archiving system, or network storage, or directly from a scanner for example. Typically, the image is an X-ray image, but may alternatively be a CT image or a MR image or another medical imaging technology. For example, the image is an image that shows the hips and pelvis of the patient. Alternatively, the image may be a of a femur, a vertebra in the spine, an ankle, a wrist, a clavicle or a mandible, or another bone or group of bones in the body.

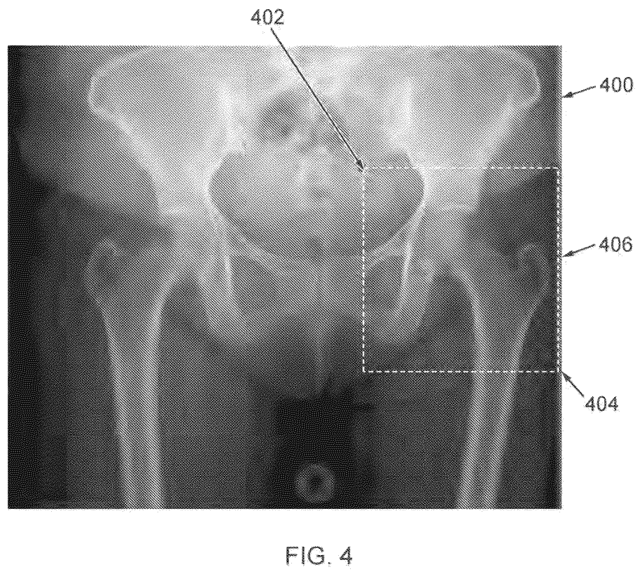

At step 302 a region of Interest of the medical image is determined. As shown in FIG. 4 the region is preferably a rectangular region within the medical image, although other shapes for the region are possible. In the system and method of analysing medical images, machine learning models are used to determine the boundaries of the region of interest. Alternatively, a convolution neural network and/or a multilayer perceptron is used to compute the boundaries of the region of interest. A machine learning or AI model may also be used to identify the boundary of the cortical bone and/or the trabecular bone.

An example is now presented for the system and method of analysing medical images in which the region of the medical image is a region containing the proximal femur (on the left and/or right hip), though another bone or bone group is possible. FIG. 4 shows a rectangular region within an X-ray image showing a hip and femur. The points 402 and 404 define opposite corners of a region 406. This region 406 is cropped from the input image 400, and thus provided a simpler, more consistent, input for the pixel level segmentations further along the pipeline 300. This cropping of the image 400 to region of interest 406 is done to remove most artefacts that may be present on the image and may cause problems with the subsequent processing. A Visual Geometry Group (VGG) network architecture is selected for this application including a CNN. The input is a pelvic or hip X-Ray, though other bones or bone groups are possible. The output is a pair of coordinates describing the top left and bottom right corners of the ROI rectangle. Pre-trained VGG network weights are available in the public domain, allowing deployment with minimal training. Alternatively, other network architectures such as ResNet, Inception, EfficientNet may be used in further configurations of the system and method for analysis of medical images.

In clinical practice, pelvic and hip X-Rays or other medical scan images, are acquired under a wide range of circumstances; as a result, they often contain artefacts such as: hip or other surgical implants, femoral or surgical nails, screws, radiation shields, catheters, and surgical drains or other foreign bodies. The foreign bodies may be internal or external to skin, and in some cases they are external bodies which may be superimposed on the area of interest like a surgical drain.

This system and method of analysing medical images allows the artefacts to be identified on the medical image. Alternatively, the artefacts may be artefacts that arise from soft tissue, skin or body fat.

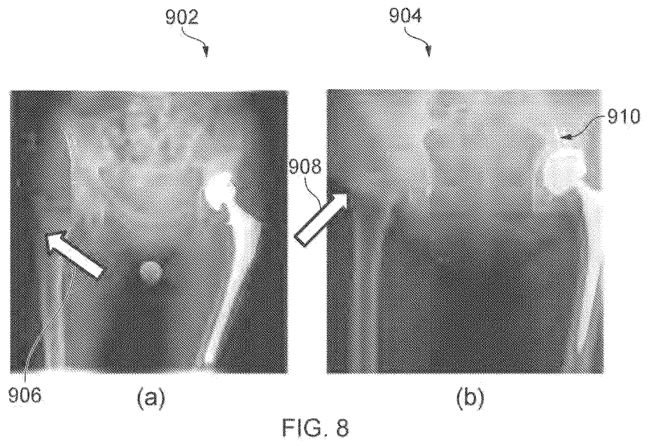

For example, FIGS. 8A and 8B show unsuccessful segmentations. FIG. 8A shows an image 802 from a patient with a low BMI (17.04) and a loose skin fold. FIG. 8B shows an image 804 from a patient with a high BMI (41.02) and an abdominal skin fold. The arrows, 806 and 808 each indicate an artefact that has arisen due to the soft tissue. FIG. 8B also shows a surgical pin 810 which may also result in an imaging artefact. The red dots also indicate opposed corners that will define the region of interest for the image.

Such artefacts, combined with significant anatomical variation due to pathologies and body types, and differences in radiographic positioning, presents a major challenge for segmentations of the X-ray image with pixel level accuracy. A convolutional neural network is used to identify the one or more artefacts, and the medical image is further processed to remove the one or more artefacts from the region of interest of the medical image.

The system configured to implement the method disclosed is based on explainable AI or ML techniques and can detect and take account of such artefacts.

After an outer boundary of the cortical bone has been identified in step 360, the outer boundary of the cortical bone that was identified in the region of interest is then segmented. Preferably, the region of cortical bone that has been identified in the region of interest is also segmented. A known machine learning method utilises multiple layers, each examining the image at a different spatial resolution. This enables it to detect large low-frequency changes in the image, such as the shape of the hip joint, as well as high-frequency details such as the different textures of bone and soft tissue.

Training the machine learning model that enables it to accurately segment commonly missed features. The outer boundary of the cortical bone is identified using the contrast between the edge of the at least one portion of bone and the surrounding area of the image.

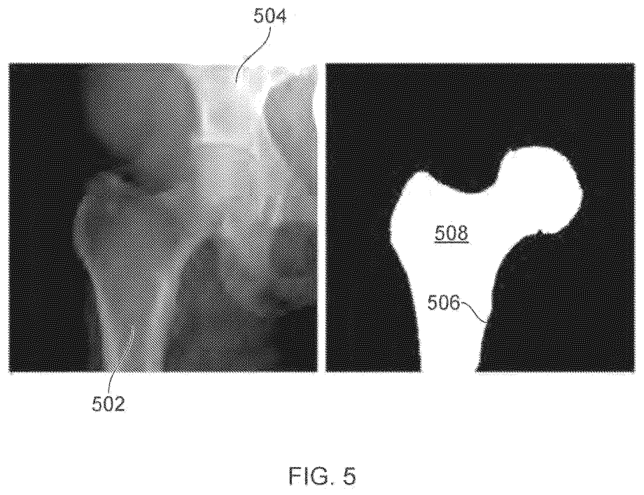

FIG. 5 shows example bone images from U-Net output on training data. Information on U-Net may be found, for example, by MICCAI 2015, pp 234-231, Part III, LNCS 9351, Ronneberger et. al.

FIG. 5 shows an X-ray image on the left side (FIG. 5A) including showing an upper portion of a femur 502 and a portion of a hip bone 504. FIG. 5 also shows on the right side an example bone image that has been segmented (FIG. 5B from the X-ray image on the left side. In FIG. 5B, the upper portion of the femur 502 is shown by the segmented bone 508. The segmentation boundary 506 of the segmented bone 508 is identified by the system and method for analysis of medical images disclosed herein.

The identified boundaries are used to determine at least one of the bone parameters for the at least one portion of bone within the region of interest.

One or more of the outputs of the segmentation steps are checked for shape acceptance against a known shape metric. Preferably, the outputs are checked for realism using an Active Shape Model (ASM). The shape metric is transformed to align with the segmented outer boundary. The transformation of the known shape metric comprises at least one of: rotation, translation, or scaling of the parameters of the known shape metric.

In some configurations of the system and method for analysis of medical images comparison of the fitted shape and the known metric is performed using two metrics: shape loss and area loss. However, the comparison may also be done with one of the two metrics. That is either the shape loss metric or the area loss metric.

Shape loss is the sum of the distances of each point in the shape to the nearest edge found in the segmentation. The shape loss metric relates a distance in the shape to a nearest edge in the segmentation. This is low when the shape tightly fits the border of the mask, and high where the mask cannot accurately be represented by a realistic proximal femur shape. Preferably, the shape loss metric is calculated as the total perpendicular distance between each of the control points of the transformed parametrised shape and the segmented area, normalised by the number of control points and image pixel spacing.

Area loss is the proportion of 0 values in the segmentation found inside the fitted active shape added to the proportion of 1s found outside the shape. The area for which the segmentation mask and fitted active shape do not agree is outside the shape. The area loss metric is relatively low for shapes that tightly match the segmentation compared to shapes that do not encompass much of the mask or those that leave large gaps inside their outline. Preferably the area loss metric is computed as the ratio of a symmetric difference of the set of pixels in a transformed parameterised shape and the segmented area of the body part, to the total number of pixels in the segmented area.

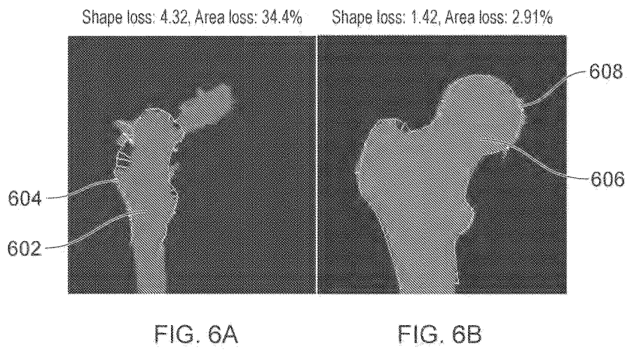

This process by which the outputs of the segmentation steps are checked for shape acceptance against a known shape metric is quantitative, reliable, and repeatable. Inaccurate segmentations are rejected for insufficient shape acceptance. Accurate segmentations that have sufficient shape acceptance are passed on to further the analysis. An example of this process is shown by FIG. 6.

On the left side of FIG. 6 is FIG. 6A which shows a poorly segmented proximal femur 602 The segmentation boundary 604 of the segmented proximal femur 602 has been determined by shape fitting.

On the right side of FIG. 6 is FIG. 6B which shows a well segmented proximal femur 606. There is a segmentation boundary 608 of the segmented proximal femur 606. The edges of the segmented proximal femur 606 are cleaned by the fitted active shape of the segmentation boundary 608.

The poorly segmented femur 602 of FIG. 6A has a shape loss of 4.32 pixels (0.622 mm), and an area loss of 34.4%. By contrast, the well fitted femur 606 of FIG. 6B has a shape loss of 1.42 pixels (0.204 mm) and an area loss of only 2.91%.

The fitted active shape cleans up the edges of a segmentation by a convolutional neural network chosen to be the final mask defining proximal femur outline. The convolutional neural network has an architecture for fast and precise segmentation of images such as but not limited to U-Net.

In some configurations of the system and method for analysis of medical images a threshold is applied to the known shape metric to identify images for which the bone segmentation is not within an allowed tolerance. For area loss the threshold is typically 10%, with a commercially accepted value between 0-30%. For the shape loss, the threshold is typically 3 pixels or 0.432 mm, with a commercially accepted value between 0-7 pixels (0.0-1.0 mm).

### Cortical Bone Segmentation and Measurement

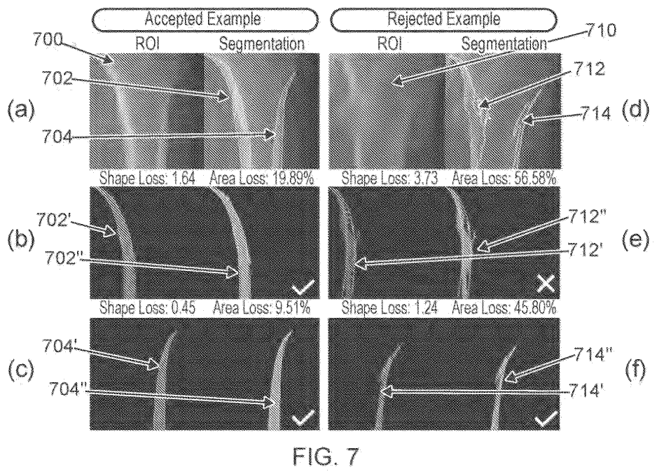

In some configurations of the system and method of analysing medical images, the identified boundaries are used to determine at least one of the bone parameters for the at least one portion of bone within the region of interest. For an example shown in FIG. 7 the region of interest includes a cortical portion of a bone in the image. The cortical portion includes the osseous tissue that forms the outermost layer of bone. FIGS. 7A, 7B, 7C, 7D, 7E, and 7F show active shapes fitted around a range of inner and outer cortical segmentations.

Segmentation of the region of bone, preferably cortical bone follows the same overall algorithmic framework as the proximal femur segmentation discussed above, although, it is preferably on a refined region of interest, although a larger region may be segmented.

To assess the plausibility that the segmented shape of the cortical portion is correct, the output of the segmentation algorithm is partitioned into separate inner and outer cortical portions so that individual active shapes are fitted. The area is determined of the at least a portion of bone in the region of interest that is occupied by cortical portion. As the area of the cortical portion is much smaller in comparison to the total area of the proximal femur, larger threshold values of the shape-loss and area-loss metrics are used to determine acceptable segmentations.

FIGS. 7A and 7D show a region of interest (ROI) 700 and 710 from a medical image (in this case an X-ray image), and the corresponding segmentations 702, 712 and 704, 714 of the ROI of the medical image. FIG. 7A shows an accepted example of segmentation and FIG. 7D shows a rejected example.

FIGS. 7B and 7C show different segmented regions for the ROI 700. In FIG. 7B, region 702′ has a shape loss of 1.64, and region 702″ has area loss of 19.98%, compared to the original image. In FIG. 7C region 704′ has a shape loss of 0.45 and region 704″ an area loss of 9.51%. FIG. 7B and FIG. 7C both show a respective accepted example of segmentation.

By contrast, the shape loss and area loss for a rejected example are much greater. FIGS. 7E and 7F show examples that would be rejected. As seen, the segmentation in the rejected example is much less smooth than the segmentation in the accepted example of FIG. 7A. FIGS. 7E and 7F show different segmented regions for the ROI 710. In FIG. 7E, region 712′ has a shape loss of 3.73, and region 712″ has area loss of 56.58%, compared to the original image. In FIG. 7F region 714′ has a shape loss of 1.24 and region 714″ an area loss of 45.8%.

The combination of the proximal femur segmentation, and the cortical bone segmentation enables accurate calculation of the proportion of the area of the proximal femur and femur shaft occupied by cortical bone. It also enables the ratio of cortical bone to trabecular bone in the at least one portion of bone in the region of interest is also determined. The ratio is preferably determined as the ratio of the area of the cortical segments and the area of the proximal femur within the inter-trochanteric region. This metric is chosen as it is found that the Bone Mineral Density (BMD) of the inter-trochanteric region in Dual Energy X-Ray Absorptiometry (DEXA) scans contribute most to the overall BMD.

### Trabecular Structure Segmentation and Quantification

### Tissue Classification

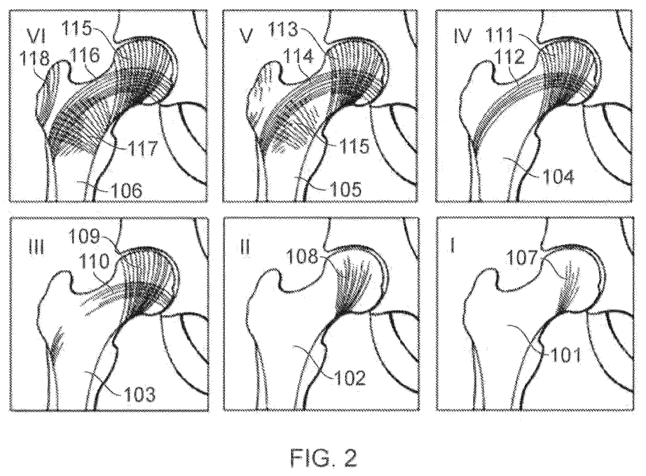

FIG. 2 shows visibility of trabeculae graded according to the Singh Index. Six illustrations of an upper femur with a ball joint in a socket of a pelvis are shown. The grade of the trabeculae according to the Singh index are shown in the upper left corner of each illustration. The Singh Index grades are I, II, III, IV, V, and VI.

The Singh Index is based on a simplified representation (and disappearance) of five specific trabecular groups as shown in FIG. 2: principal compressive, secondary compressive, primary tensile, secondary tensile, and intertrochanteric.

In the illustration in FIG. 2 showing Singh Index grade I, the upper femur 101 has only principle compressive trabeculae 107 visible. In the illustration showing grade II, the upper femur 102 has principle compressive trabeculae 108 visible, while other trabeculae are nearly resorbed. In the illustration showing grade III, there are principle compressive trabeculae 109 visible in the upper femur 103, and there are principle tensile trabeculae 110 thinned with breakage in continuity.

In the illustration in FIG. 2 showing Singh Index grade IV, the upper femur 104 has principle compressive trabeculae 111 visible, and principle tensile trabeculae 112 thinned without loss of continuity. In the illustration showing grade V, the upper femur 105 has principle compressive trabeculae 113 visible and principle tensile trabeculae 114 visible and prominence of trabeculae in a Ward triangle 115. In the illustration showing grade VI, the upper femur 106 all trabeculae 115, 116, 117, 118 visible and of normal thickness.

In the system and method of analysing medical images disclosed herein, the ratio of cortical to trabecular bone is used to determine one or more of the at least one bone parameters of bone mineral density (e.g. using cortical ratios), trabecular features (e.g. orientation, length, width, and like characteristics of trabeculae).

In the system and method of analysing medical images disclosed herein, the one or more bone parameters are used to determine one of more actionable metrics. Preferably, wherein the actionable metrics comprises at least one of:
an indication of the presence of osteoporosis/osteopenia;
a classification of osteoporosis, osteopenia or healthy bone structure in line with that expected in the general population;
is a prediction of the severity of osteoporosis;
a risk of fracture in a given period;
a prediction of intra-operative fracture;
a proposal of a suitable bone implant type; and
a prediction of surgical outcomes, such as peri-prosthetic fracture and aseptic loosening following insertion of a bone implant.

Further preferably, the actionable metric is determined to take account of one or more of: age, sex, ethnicity, height, weight, BMI, smoking status, alcohol use, Glucocorticoid use, prior fracture, age at menarche, age at menopause and family history.

The system and method of analysing medical images disclosed may be configured whereby the actionable metric further comprises a set of individual bone health metrics from imaging examinations acquired at different points in time.

A final cortical feature set (used to build an osteoporosis classifier) includes the ratio of the area of the region occupied by cortical bone. This metric was chosen as it was found that the Bone Mineral Density (BMD) of the inter-trochanteric region in Dual Energy X-Ray Absorptiometry (DEXA) scans contributed most to the overall BMD. This is unsurprising as it occupies the largest proportion of the DEXA volume.

### Prediction of Clinically-Actionable Metrics

The system and method of analysing medical images may be configured whereby the cortical and trabecular features are used in their own right to provide information about bone health. In another embodiment of the invention, the cortical and trabecular features are used in a multi-variate statistical model to predict pathology states and other clinically-actionable metrics, e.g. the prediction of bone disease, peri-prosthetic fracture.

The system and method of analysing medical images disclosed here exploits the significantly higher resolution of X-Ray compared to Dual Energy X-Ray Absorptiometry (DEXA), to
1. extract and quantify one or more features of bone (including features of bone quantity, quality and morphometry) that are independently associated with bone strength, including:
   Measurement of the composition of the overall bone structure in terms of the underlying constituent bone types (cortical bone and trabecular bone), relative amounts of the two types, including thickness, density, and morphometry, for example size and shape.
   Measurement of the segmentation of the trabeculae within the trabecular bone that contribute to the bones overall biomechanical strength.
   Measurement of the architectural pattern of the cortical and trabecular bone that contribute to the bones overall biomechanical strength.
2. identify osteopenia, osteoporosis and other metabolic bone disorders which result in reduced bone strength, including at earlier stages.
3. Identify other clinically-actionable metrics, such as prediction of post-operative outcomes and optimal therapeutic agent.

The invention has been described by way of examples. Since numerous modifications will readily occur to those skilled in the art, it is not desired to limit the invention to the exact construction and operation shown by the figures and presented in the description. Accordingly, all suitable modifications and equivalents may be resorted to which fall within the scope of the claims.

In the claims, any reference signs placed between parentheses shall not be construed as limiting. The word ‘comprising’ does not exclude the presence of other elements or steps then those listed. The terms ‘a’ or ‘an,’ as used herein, are defined as one or more than one. The use of phrases such as ‘at least one’ and ‘one or more’ in the claims should not be construed to imply that the introduction of another claim element by the indefinite articles ‘a’ or ‘an’ limits any particular claim containing to inventions containing only one such element, even when the same claim includes the introductory phrases ‘one or more’ or ‘at least one’ and indefinite articles such as ‘a’ or ‘an.’ The mere fact that certain measures are recited in mutually different claims does not indicate that a combination of these measures cannot be used to advantage.

1. A method of analysing a medical image showing at least a portion of a bone, comprising steps of: determining a region of interest of the medical image; identifying a boundary of at least one of an inner edge and an outer edge of cortical bone of the at least a portion of the bone within the region of interest; identifying at least one boundary of trabecular bone of the at least a portion of the bone within the region of interest; utilising the identified boundaries to determine at least one bone parameter including: orientation, bone mineral density, length, and width of trabecular structure, for the at least a portion of the bone within the region of interest.

2. A method as claimed in claim 1 wherein the medical image is one of: an X-ray image, a CT scan, an MR image.

3. (canceled)

4. (canceled)

5. (canceled)

6. (canceled)

7. (canceled)

8. A method as claimed in claim 1 wherein after the boundary of the outer edge of the cortical bone has been identified, segmenting the cortical bone contained within the identified boundary of the outer edge within the region of interest to determine a segmentation mask, checking the segmentation mask for shape acceptance using a known shape metric transformed to align with the boundary of the outer edge of the cortical bone by at least one of at least one of: rotation, translation, scaling, of shape parameters of the known shape metric, wherein the known shape metric comprises at least one of a shape loss metric which relates a distance from a fitted active shape to a nearest edge of the segmentation mask, and an area loss metric which is a proportion of zero values in the segmentation mask inside the fitted active shape.

9. A method as claimed in claim 8 wherein the area loss metric is computed as a ratio of a symmetric difference of a set of pixels in the fitted active shape and the segmentation mask, to a total number of pixels in segmentation mask.

10. A method as claimed in claim 9 wherein the shape loss metric is calculated as a total perpendicular distance between each of a plurality of control points of the fitted active shape and the segmentation mask and normalised by the number of the plurality of control points and an image pixel spacing.

11. A method as claimed in claim 10 wherein a threshold is applied to the known shape metric to identify images for which segmentation is not within an allowed tolerance.

12. A method as claimed in claim 11 further comprising a step of determining an area of the at least a portion of bone in the region of interest that is occupied by cortical bone, and determining a ratio of cortical to trabecular bone in the region of interest wherein the ratio of cortical to trabecular bone is used to determine one or more of the at least one bone parameter.

13. (canceled)

14. (canceled)

15. (canceled)

16. A method as claimed in claim 12 wherein the outer boundary of the cortical bone is identified using a contrast between an edge of the at least a portion of the bone and a surrounding area of the medical image.

17. A method as claimed in claim 12 wherein a first machine learning model is used to identify the identified boundaries of the cortical bone and the trabecular bone, and a second machine learning model is used to determine one or more ROI boundaries of the region of interest.

18. (canceled)

19. (canceled)

20. A method as claimed in claim 12 further comprising the step of determining a verification metric to assess plausibility of the segmentation mask in the region of interest compared to a database of predefined segmented shapes.

21. A method as claimed in claim 12 further comprising a step of identifying imaging artefacts in the region of interest of the medical image.

22. A method as claimed in claim 21 wherein the artefacts comprise one or more of: a surgical implant, a surgical nail, or other internal or external foreign body.

23. A method as claimed in claim 21 wherein the artefacts are artefacts arising from soft tissue, skin or body fat.

24. A method as claimed in claim 21 wherein a convolutional neural network is used to identify the artefacts, and the medical image is further processed to remove the one or more artefacts from the region of interest of the medical image.

25. A method as claimed in claim 12 wherein the at least a portion of the bone comprises at least one of: femur, vertebrae, ankle, wrist, clavicle, mandible.

26. (canceled)

27. A method as claimed in claim 12 wherein the one or more bone parameters are used to determine one of more actionable metrics comprising at least one of: an indication of a presence of osteoporosis/osteopenia; a classification of osteoporosis, osteopenia or healthy bone structure in line with that expected in a general population; is a prediction of a severity of osteoporosis; a risk of fracture in a given period; a prediction of intra-operative fracture; a proposal of a suitable bone implant type; is a prediction of surgical outcomes, such as peri-prosthetic fracture and aseptic loosening following insertion of a bone implant.

28. (canceled)

29. (canceled)

30. A system configured to implement the method according to claim 1.
